(19) United States
(12) Patent Application Publication
(10) Pub. No.: US 2005/0065203 A1
(43) Pub. Date: Mar. 24, 2005

(54) Method of reducing type 2 diabetes in high risk patients

(76) Inventor: Salim Yusuf, Frankfurt (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/492,919

(22) PCT Filed: Oct. 17, 2002
(86) PCT No.: PCT/EP02/11636

Related U.S. Application Data
(60) Provisional application No. 60/344,495, filed on Oct. 17, 2001.

(51) Int. Cl. A61K 31/401

(52) U.S. Cl. 514/423

(57) ABSTRACT

A method of reducing diabetes in patients who are at risk for developing diabetes comprising administering to such patients an effective amount of an angiotensin converting enzyme (ACE) inhibitor for a sufficient period of time to prevent the development of diabetes in such patients; a method of slowing or reversing the decline of β-cell function in an individual comprising administering to an individual an effective amount of an ACE inhibitor; a method of increasing islet blood flow in an individual comprising administering to an individual an effective amount of an ACE inhibitor; a method of increasing pancreatic β-cell perfusion in an individual comprising administering to an individual an effective amount of an ACE inhibitor and a method of lowering aldosterone secretion and renal potassium wasting in an individual by comprising administering to an individual an effective amount of an ACE inhibitor.

### FIELD OF INVENTION

The present invention relates to a method of reducing diabetes in patients who are at risk for developing diabetes comprising administering to such patients an effective amount of an angiotensin converting enzyme (ACE) inhibitor for sufficient period of time to prevent the development of diabetes in such patients; to a method of slowing or reversing the decline of β-cell function in an individual comprising administering to an individual an effective amount of an angiotensin converting enzyme inhibitor for a sufficient period of time to prevent the decline of β-cell function in such individual; a method of increasing islet blood flow in an individual comprising administering to an individual an effective amount of an angiotensin converting enzyme inhibitor for a sufficient period of time to increase islet blood flow in such individual; a method of increasing pancreatic β-cell perfusion in an individual comprising administering to an individual an effective amount of an angiotensin converting enzyme inhibitor for a sufficient period of time to increase pancreatic β-cell perfusion in such individual and a method of lowering aldosterone secretion and renal potassium wasting in an individual by comprising administering to an individual an effective amount of an angiotensin converting enzyme inhibitor for a sufficient period of time to lower aldosterone secretion and renal potassium wasting in such individual.

The present invention further relates to the use of an angiotensin converting enzyme (ACE) inhibitor or a pharmaceutically acceptable derivative thereof in the manufacture of a medicament for the prevention or reduction of the onset of diabetes in patients who are at risk for developing diabetes; for the prevention, slowing or reversing the decline of β-cell function; for increasing islet blood flow; for increasing pancreatic β-cell perfusion; and for lowering aldosterone secretion and renal potassium wasting.

### BACKGROUND OF THE INVENTION

ACE inhibitors are well known in the art for their activity in inhibiting angiotensin converting enzyme, thereby blocking conversion of the decapeptide angiotensin I to angiotensin II. The principal pharmacological and clinical effects of ACE inhibitors arise from suppression of synthesis of angiotensin II. Angiotensin II is a potent pressor substance and, therefore, blood pressure lowering can result from inhibition of its biosynthesis, especially in animals and humans whose hypertension is angiotensin II related. ACE inhibitors are effective antihypertensive agents in a variety of animal models and are clinically useful for the treatment of hypertension in humans. ACE inhibitors are also employed for the treatment of heart conditions such as congestive heart failure.

It has been found that ACE inhibitors are also useful for the prevention of diabetes in patients that are at high risk for developing diabetes.

The present invention relates to a method of reducing diabetes in patients who are at risk for developing diabetes comprising administering to such patients an effective amount of an angiotensin converting enzyme (ACE) inhibitor for sufficient period of time to prevent the development of diabetes in such patients; to a method of slowing or reversing the decline of β-cell function in an individual comprising administering to an individual an effective amount of an angiotensin converting enzyme inhibitor for a sufficient period of time to prevent the decline of β-cell function in such individual; a method of increasing islet blood flow in an individual comprising administering to an individual an effective amount of an angiotensin converting enzyme inhibitor for a sufficient period of time to increase islet blood flow in such individual; a method of increasing pancreatic β-cell perfusion in an individual comprising administering to an individual an effective amount of an angiotensin converting enzyme inhibitor for a sufficient period of time to increase pancreatic β-cell perfusion in such individual and a method of lowering aldosterone secretion and renal potassium wasting in an individual by comprising administering to an individual an effective amount of an angiotensin converting enzyme inhibitor for a sufficient period of time to lower aldosterone secretion and renal potassium wasting in such individual.

The present invention further relates to the use of an angiotensin converting enzyme (ACE) inhibitor or a pharmaceutically acceptable derivative thereof in the manufacture of a medicament for the prevention or reduction of the onset of diabetes in patients who are at risk for developing diabetes; for the prevention, slowing or reversing the decline of β-cell function; for increasing islet blood flow; for increasing pancreatic β-cell perfusion; and for lowering aldosterone secretion and renal potassium wasting.

Type 2 diabetes is an important and common risk factor for the development of coronary artery disease, strokes, peripheral arterial disease, and renal and eye disease. Currently, in North America, the direct and indirect costs of diabetes and its complications exceeds $100 billion per year. This health and economic impact of diabetes is bound to increase, as the global prevalence of diabetes rises from 4.2% to 5.4% by the year 2025.

A growing amount of literature shows that the complications of diabetes can be reduced or prevented by improving glucose control (N. Engl. J. Med. 1993; 329: 977-986; Lancet, 1998; 352: 837-852), lowering blood pressure (BMJ, 1998; 317: 713-720) and lipids (Lancet, 1994, 344: 1383-1389), smoking cessation, and taking angiotensin converting enzyme (ACE) inhibitors (Lancet, 2000, 255; 253-259). An even more effective approach to preventing these problems would be to prevent diabetes from developing. Whereas recent evidence from trials suggests that lifestyle modifications may reduce the risk of diabetes (Diabetes Care, 1997, 20, 537-544), the long-term adherence to such interventions has not been high. Therefore alternative strategies that are more easily implemented, safe ad likely to prevent not only diabetes but also its chronic consequences deserve to be investigated. Recently, it was demonstrated that the ACE inhibitor, ramipril, reduces myocardial infarction, strokes, death, and the development of diabetic nephropathy among people at high risk, both with and without a diagnosis of diabetes (Lancet, 2000, 255; 253-259; N Engl J. Med. 2000; 342: 145-153). It was also observed that ramipril reduced the development of diabetes in study participants without known diabetes at randomization (N Engl J. Med. 2000; 342: 145-153).

The phrase “diabetes” as used herein includes both type I diabetes, also known as insulin-dependent, diabetes mellitus (IDMM), and type II diabetes, also known as non-insulin-dependent diabetes mellitus (NIDDM).

The phrase “angiotensin converting enzyme inhibitor” (“ACE inhibitor”) is intended to embrace an agent or compound, or a combination of two or more agents or compounds, having the ability to block, partially or completely, the rapid enzymatic conversion of the physiologically inactive decapeptide form of angiotensin (“Angiotensin I”) to the vasoconstrictive octapeptide form of angiotensin (“Angiotensin II”).

Example of ACE inhibitors suitable for use herein are for instance the following compounds: AB-103, ancovenin, benazeprilat, BRL-36378, BW-A575C, CGS-13928C, CL242817, CV-5975, Equaten, EU-4865, EU4867, EU-5476, foroxymithine, FPL 66564, FR-900456, Hoe065, 15B2, indolapril, ketomethylureas, KR1-1177, KR1-1230, L681176, libenzapril, MCD, MDL-27088, MDL-27467A, moveltipril, MS-41, nicotianamine, pentopril, phenacein, pivopril, rentiapril, RG-5975, RG-6134, RG-6207, RGH0399, R00-911, RS-10085-197, RS-2039, RS 5139, RS 86127, RU44403, S-8308, SA-291, spiraprilat, SQ26900, SQ-28084, SQ-28370, SQ-28940, SQ-31440, Synecor, utibapril, WF-10129, Wy44221, Wy-44655, Y-23785, Yissum, P-0154, zabicipril, Asahi Brewery AB-47, alatriopril, BMS 182657, Asahi Chemical C-111, Asahi Chemical C-112, Dainippon DU-1777, mixanpril, Prentyl, zofenoprilat, 1 (-(1-carboxy-6-(4-piperidinyl) hexyl) amino)-1-oxopropyl octahydro-1H-indole-2-carboxylic acid, Bioproject BP1.137, Chiesi CHF 1514, Fisons FPL66564, idrapril, perindoprilat and Servier S-5590, alacepril, benazepril, captopril, cilazapril, delapril, enalapril, enalaprilat, fosinopril, fosinoprilat, imidapril, lisinopril, perindopril, quinapril, ramipril, ramiprilat, saralasin acetate, temocapril, trandolapril, trandolaprilat, ceranapril, moexipril, quinaprilat and spirapril.

A group of ACE inhibitors of high interest are alacepril, benazepril, captopril, cilazapril, delapril, enalapril, enalaprilat, fosinopril, fosinoprilat, imidapril, lisinopril, perindopril, quinapril, ramipril, ramiprilat, saralasin acetate, temocapril, trandolapril, trandolaprilat, ceranapril, moexipril, quinaprilat and spirapril.

Of particular interest is the ACE inhibitor ramipril.

Pharmaceutically acceptable derivatives of ACE inhibitors are understood to include physiologically tolerable salts of ACE inhibitors, such physiologically tolerable salts are understood as meaning both their organic and inorganic salts, such as are described in Remington's Pharmaceutical Sciences (17th Edition, page 1418 (1985)). On account of the physical and chemical stability and the solubility, for acidic groups, inter alia, sodium, potassium, calcium and ammonium salts are preferred; for basic groups, inter alia, salts of hydrochloric acid, sulfuric acid, phosphoric acid or of carboxylic acids or sulfonic acids, such as, for example, acetic acid, citric acid, benzoic acid, maleic acid, fumaric acid, tartaric acid and p-toluenesulfonic acid are preferred.

The ACE inhibitors suitable for use herein or their pharmaceutically acceptable derivatives can be used in animals, preferably in mammals, and in particular in human, as pharmaceuticals per se, in mixtures with one another or in the form of pharmaceutical preparations.

The present invention also relates to pharmaceutical formulations comprising as active ingredient at least one ACE inhibitor and/or an pharmaceutically acceptable derivative thereof in addition to customary pharmaceutically innocuous excipients and auxiliaries and their use in the prevention of diabetes or the decline of β-cell function, the increasing of islet blood flow or pancreatic β-cell perfusion and lowering aldosterone secretion and renal potassium wasting and the production of medicaments therefor. The pharmaceutical preparations normally contain 0.1 to 99 percent by weight, preferably 0.5 to 95 percent by weight, of the ACE inhibitor and/or an pharmaceutically acceptable derivative thereof. The pharmaceutical preparations can be prepared in a manner known per se. To this end, the ACE inhibitor and/or an pharmaceutically acceptable derivative thereof are brought, together with one or more solid or liquid pharmaceutical excipients and/or auxiliaries and, if desired, in combination with other pharmaceutical active compounds into a suitable administration form or dose form, which can then be used as a pharmaceutical in human medicine or veterinary medicine.

Pharmaceuticals which contain an ACE inhibitor and/or an pharmaceutically acceptable derivative thereof can be administered orally, parenterally, intravenously, rectally or by inhalation, the preferred administration being dependent on the particular symptoms of the disorder. The ACE inhibitors and/or an pharmaceutically acceptable derivative thereof can be used here on their own or together with pharmaceutical auxiliaries, namely both in veterinary and in human medicine.

The person skilled in the art is familiar on the basis of his expert knowledge with the auxiliaries, which are suitable for the desired pharmaceutical formulation. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers or colorants.

For an oral administration form, the active compounds are mixed with the additives suitable therefor, such as excipients, stabilizers or inert diluents and are brought by means of the customary methods into the suitable administration forms, such as tablets, coated tablets, hard'capsules, aqueous, alcoholic or oily solutions. Inert excipients which can be used are, for example, gum arabic, magnesia, magnesium carbonate, potassium phosphate, lactose, glucose or starch, in particular corn starch. Preparation can take place here both as dry and as moist granules. Possible oily excipients or solvents are, for example, vegetable or animal oils, such as sunflower oil or codliver oil.

For subcutaneous or intravenous administration, the active compounds are brought into solution, suspension or emulsion, if desired with the substances customary therefor such as solubilizers, emulsifiers or other auxiliaries. Suitable solvents, for example, are: water, physiological saline solution or alcohols, e.g. ethanol, propanol, glycerol, and additionally also sugar solutions such as glucose or mannitol solutions, or alternatively a mixture of the various solvents mentioned.

Pharmaceutical formulations suitable for administration in the form of aerosols or sprays are, for example, solutions, suspensions or emulsions of the active compound of the formula I in a pharmaceutically acceptable solvent, such as, in particular, ethanol or water, or a mixture of such solvents.

If required, the formulation can also contain other pharmaceutical auxiliaries such as surfactants, emulsifiers and stabilizers, and also a propellant. Such a preparation customarily contains the active compound in a concentration from approximately 0.1 to 10, in particular from approximately 0.3 to 3, % by weight.

The dose of the active compound to be administered and the frequency of administration will depend on the potency and duration of action of the compounds used; additionally also on the nature of the indication and on the sex, age, weight and individual responsiveness of the mammal to be treated.

On average, the daily dose in a patient weighing approximately 75 kg is at least 0.001 mg/kg, preferably 0.01 mg/kg, to about 20 mg/kg, preferably 1 mg/kg, of body weight.

The ACE inhibitors and/or an pharmaceutically acceptable derivative thereof can also be used to achieve an advantageous theraupeutic action together with other pharmacologically active compounds for the prevention of the above-mentioned syndromes.

The following is a description of a clinical trial employing the ACE inhibitor ramipril to exemplify the methods of the present invention.

Methods

The design of the Heart Outcomes Prevention Evaluation (HOPE) trial has been described in detail in previous publications. Briefly, individuals who were 55 years or older with no evidence of left ventricular dysfunction or heart failure and who had evidence of vascular disease or who had diabetes and one other risk factor were eligible as long as they had no indication or contraindication to receiving an ACE-inhibitor. The study was conducted in 26 hospitals in 19 countries from 1994 to 1999. All patients provided written informed consent.

Of 10576 eligible patients who participated in a run-in period during which they received 2.5 mg ramipril once daily for 1 week followed by matching placebo for 10 to 14 days, 1035 (9.8%) were excluded from randomization (3.2% for side effects, 3.7% for lack of consent). Of the remaining 9541 patients, 3654 (38.3%) had a clinical diagnosis and 5887 (61.7%) did not at randomization. In the following it is primarily foccuessed on the latter group of patients. Of these patients, 5720 were randomized to receive up to 10 mg of ramipril once per day or equivalent placebo. One hundred sixty-seven patients who were randomized to receive a low dose (2.5 mg/day) of ramipril as part of the Study to Evaluate Carotid Ultrasound changes with Ramipril and Vitamin E (SECURE). Substudy results are not included. All randomized patients were also randomized to receive 400 IU of Vitamin E or placebo.

Follow-up visits occurred at 1 month and 6 months after randomization and then every 6 months (mean follow-up of 4.5 years). At each visit, it was documented whether the diagnosis of diabetes had been made since the last visit.

The primary outcome of this analysis is a new diagnosis of diabetes recorded on the basis of self-report. This diagnoses was made blinded to treatment allocation and, hence, is likely to be unbiased. Hemoglobin A_{1c} (HbA_{1c}) levels and medications used among those diagnosed as having diabetes were also recorded. The HbA_{1c} levels; were determined locally. Values higher than 110% of the upper limit of normal for each laboratory were considered to be biochemical confirmation of diabetes.

Statistical Analysis

Survival curves utilizing the Kaplan Meier and log-rank procedures were used to describe and compare the results in the 2 treatment groups. Because of the factorial design, all analyses were stratified for randomization to vitamin E or placebo. Subgroup analyses were conducted using tests of interaction in the Cox regression model.

Results

The baseline characteristics of the patients who did not have diabetes are provided in Table 1.

TABLE 1
Baseline Demographics in Patients Without Diabetes Who Entered into
HOPE

| Characteristics | Remipril Group | Placebo Group |
|---|---|---|
| Total No. randomized | 2837 | 2883 |
| | Mean (SD) | |
| Age | 68.3 (6.7) | 65.9 (6.9) |
| Blood pressure, mm HG | | |
| Systolic | 136.4 (19.5) | 136.7 (19.4) |
| Diastolic | 78.2 (10.5) | 78.7 (10.5) |
| Heart rates, beats/min | 66.2 (10.8) | 66.5 (10.8) |
| Body Mass Index | 26.9 (3.9) | 27.2 (4.0) |
| | No. (%) | |
| Women | 583 (20.5) | 575 (19.9) |
| Nonwhites | 233 (8.21) | 239 (8.29) |
| Waist-hip ratio | 0.93 (0.08) | 0.93 (0.08) |
| Coronary artery disease | 2645 (93.2) | 2693 (93.4) |
| Myocardial infarction | 1784 (62.9) | 1819 (63.1) |
| Stable, angina | 1826 (64.4) | 1849 (64.1) |
| Unstable angina | 861 (30.3) | 852 (29.6) |
| Stroke or transient ischemic | 347 (12.2) | 318 (11.0) |
| stroke | | |
| Peripheral arterial disease | 1106 (39.0) | 1150 (39.9) |
| Coronary artery bypass | 871 (30.7) | 881 (30.6) |
| graft surgery | | |
| Percutaneous coronary | 648 (22.8) | 624 (21.6) |
| intervention | | |
| Hypertension* | 1225 (43.2) | 1256 (43.6) |
| Cholesterol mg/dl** | 1862 (65.8) | 1928 (66.9) |
| Total > 200.8 | | |
| High-density lipoprotein | 472 (16.6) | 533 (18.5) |
| 34.7 | | |
| Current smoking | 371 (13.1) | 404 (14.0) |
| β-Blockers | 1310 (46.2) | 1348 (46.8) |
| Lipid-lowering agents | 909 (32.0) | 950 (33.0) |
| Diuretics | 363 (12.8) | 356 (12.3) |
| Calcium-channel blockers | 1376 (48.5) | 1427 (49.5) |
| Left ventricular hypertrophy | 226 (8.0) | 250 (8.7) |
| Microalbuminuria | 402 (14.2) | 421 (14.6) |

*History or blood pressure greater than 140/90 mm Hg.
**To convert total and high-density lipoprotein cholesterol from mg/dl to mmol/L, multiply by 0.0259]

The proportion of patients taking study ramipril or open label ACE-inhibitors in the active group was 98.3% at 2 years and 89.7% at 4 years. The proportion taking open label ACE-inhibitors in the control group was 11.6% and 27.4% respectively

New Diagnosis of Diabetes

There were 102 individuals (3.6%) in the ramipril group compared with 155 (5.4%) in the placebo group (relative risk [RR], 0.66; 95% confidence interval [Cl], 0.51-0.85; P<0.001) who reported a new diagnosis of diabetes. The proportion of patients diagnosed to have diabetes and a documented glycated hemoglobin of 110% or more above the upper limit of normal (1.8% vs. 3.0%; RR, 0.60; 95% Cl, 0.43-0.85; P=0.003}, those receiving an oral glucose lowering agent or insulin (2.1% vs. 3.6%; RR, 0.56; 95% Cl, 0.41-0.77; P<0.001) Those with all criteria (1.3% vs. 2.5%; RR, 0.51; 95% Cl, 0.34-0.76; P<0.001) were significantly lower in the ramipril group compared with the placebo group. Vitamin E and placebo did not differ in their effect on diabetes.

Sensitivity Analysis

Because ramipril reduced the risk of cardiovascular events and diabetic nephropathy, it was assessed whether the higher occurrence of these clinical events in placebo-treated patients increased the likelihood of ascertainment of diabetes in this group. Similar stratified analyses by the occurrence of other outcomes was also examined. As noted in TABLE 2 the impact of ramipril on the development of diabetes could not be explained by any confounding factor such as preferential ascertainment in one group vs. the other or use of concomitant medications.

TABLE 2
Effect of Ramipril on the Development of Diabetes Using a Range of
Criteria and Stratified by the Occurrence of Specific Events*

| | | | | |
|---|---|---|---|---|
| Variables | Ramipril | Placebo | RR (85%) CI) | P Value |

New Diabetes†

| | | | | |
|---|---|---|---|---|
| With primary event | 9 (2.4) | 28 (5.5) | 0.48 (0.21-0.98) | .04 |
| No primary event | 93 (3.8) | 129 (5.4) | 0.69 (0.53-0.91) | .007 |
| With new MA or | 20 (5.8) | 36 (8.4) | 0.65 (0.38-1.12) | .12 |
| ON | | | | |
| No new MA or ON | 82 (3.3) | 119 (4.9) | 0.67 (0.51-0.89) | .005 |

New Diabetes With Glycated Hemoglobulin ≧ 110%, ULN‡

| | | | | |
|---|---|---|---|---|
| With primary event | 7 (1.9) | 16 (3.4) | 0.59 (0.24-1.43) | .23 |
| No primary event | 45 (1.8)( | 71 (3.0) | 0.61 0.42-0.89) | .009 |
| With new MA or | 10 (2.8) | 25 (5.8) | 0.47 (0.23-0.98) | .04 |
| ON | | | | |
| No new MA or ON | 42 (1.7) | 62 (2.6) | 0.66 (0.48-0.98) | .04 |

New Diabetes With Oral Agents or Insulin§

| | | | | |
|---|---|---|---|---|
| With primary event | 5 (1.3) | 16 (3.4) | 0.42 (0.15-1.14) | .08 |
| No primary event | 54 (2.2) | 89 (3.7) | 0.58 (0.42-0.82) | .002 |
| With new NA or | 14 (3.9) | 27 (6.3) | 0.61 (0.32-1.16) | .13 |
| ON | | | | |
| No new MA or ON | 45 (1.8) | 78 (3.2) | 0.56 (0.39-0.81) | .002 |

New Diabetes With Oral Agents or insulin and Glycated Hemoglobulin ≧
110%, ULN II

| | | | | |
|---|---|---|---|---|
| With primary event | 4 (1.1) | 10 (2.1) | 0.54 (0.17-1.72) | .29 |
| No primary event | 32 (1.3) | 61 (2.5) | 0.51 (0.33-0.78) | .001 |
| With new MA or | 6 (1.7) | 2 (54.1) | 0.32 (0.13-0.79) | .009 |
| ON | | | | |
| No new MA or ON | 30 (1.2) | 49 (2.0) | 0.60 (0.38-0.94) | .03 |

* RR indicates relative risk; CI, confidence interval; MA, microalbuminuria; ON, overt nephropathy; ULN, upper limits of normal; and primary event, death, myocardial infarction, or stroke.
† Controlling for primary events and development of MA or ON, new diabetes with glycated hemoglobin ≧ 110% had a 0.67 RR (95% CI, 0.52-0.86).
‡ Controlling for primary events and development of MA or ON, new diabetes with glycated hemoglobin ≧ 110% had a 0.62 RR (95% CI, 0.44-0.88).
§ Controlling for primary events and development of MA or ON, new diabetes with patient taking glucose-lowering therapy hat a 0.58 RR (95% CI, 0.42-0.79).
II Controlling for primary events and development of MA or ON, new diabetes with elevated glycated hemoglobin or receiving treatment hat a 0.52 RR (95%, CI, 0.35-0.78).

Subgroup Analysis

TABLE 3 demonstrates the results among subgroups of patients with different risk factors for developing diabetes. The results are consistent among those with a waist to hip ratio below or above the median of 0.93 or less or higher than 0.93 and consistent among those with a body mass index (BMI) of 27.7 or less or higher than 27.7, those with or without a history of hypertension, those receiving or not receiving p-blockers or diuretics at randomization. A higher proportion of individuals without diabetes who were randomized to the placebo group than those randomized to the ramipril group received diuretics or β-blockers (drugs that are associated with glucose intolerance or diabetes) during the study. However, the RR for diabetes in the subgroup of individuals who never took these drugs during the study was consistent with the overall results (RR, 0.62; 95% Cl, 0.43-0.90).

TABLE 3
Effect of Ramipril in Preventing Diabetes in Subgroups Defined at Randomization

| | Ramipril | | | Pacebo | | | |
|---|---|---|---|---|---|---|---|
| | Patients | No. | (%) | Patients | No. | (%) | |
| | With | | | With | | | Interaction |
| | Diabetes | | | Diabetes | | | P Value |
| Waist-hip ratio >0.93 | 1445 | 63 | (4.4) | 1508 | 104 | (6.9) | .46 |
| Waist-hip ratio ≦0.93 | 1392 | 39 | (2.8) | 1375 | 51 | (3.7) | |
| Body mass index >27.7 Kg/m² | 1095 | 63 | (5.8) | 1146 | 94 | (8.2) | .79 |
| Body mass index ≦7.7 Kg/m² | 1742 | 39 | (2.2) | 1737 | 61 | (3.5) | |
| With hypertension | 1167 | 49 | (4.2) | 1192 | 76 | (6.4) | .86 |
| Without hypertension | 1670 | 53 | (3.2) | 1691 | 79 | (4.7) | |
| With micmalbuminuria | 402 | 17 | (4.2) | 421 | 35 | (8.3) | .26 |
| Without miaoa)buminuria | 2435 | 85 | (3.5) | 2462 | 120 | (4.9) | |
| Taking β-blocker | 1310 | 52 | (4.0) | 1348 | 66 | (4.9) | .15 |
| Not taking β-blocker | 1527 | 50 | (3.3) | 1535 | 89 | (5.8) | |
| Taking diuretics | 363 | 18 | (5.0) | 356 | 28 | (7.9) | .86 |
| Not taking diuretics | 2474 | 84 | (3.4) | 2527 | 127 | (5.0) | |

Change in Weight

In 4074 patients weight was recorded at baseline and at study end. Weight increased by a mean (SD) of 0.98 (6.93) kg in the active group and 0.76 (8.10) kg in the control group.

These analyses indicate that ramipril reduced the risk of new diagnoses of diabetes among individuals with no previous history of diabetes. The magnitude of the benefit appears to be large and moreover, ACE-inhibitors also reduce macrovascular and microvascular complications of diabetes (Lancet, 2000; 255; 253-259). Although the data on new diagnoses of diabetes were collected prospectively in the HOPE study, it was not a primary or secondary outcome of the trial. Therefore the results should be interpreted with caution. Nevertheless, the results are plausible given the clear statistical significance and consistency of results across subgroups, as well as using a range of approaches to diagnosing diabetes.

External Data

The Study to Evaluate Carotid Ultrasound changes in patients treated with Ramipril and vitamin E (SECURE) in which fasting glucose increased more with placebo (15.8 mg/dL [0.41 mmols]) than with ramipril (9.6 mg/dL [0.25 mmols]; P=0.03) (Can. J. Cardiol. 2000; 16 (suppl F) 233F). Among the patients with diabetes in the HOPE study, there was a significant reduction in HbA₁₀ levels during serial annual recordings occurred during the first 2 years (absolute difference, 0.2%) (Lancet, 2000; 255; 253-259). In the UK Prospective Diabetes Study (UKPDS) (BMJ 1998; 3, 17: 713-720) and in the Captopril Prevention Project (Lancet, 1999 353: 611-616), patients randomized to receive ACE inhibitors had lower levels of HbA₁, or less development of diabetes compared with those taking β-blockers or diuretics. However, it is not clear whether the differences in development of diabetes observed in these studies are due to a protective effect of ACE inhibitors or an adverse effect of β-blockers or diuretics.

Mechanisms of Action

Hypokalemia substantially impairs the insulin secretory response to glucose (J Cardiovasc Pharmacol. 1994; 24 (suppl 3) S61-S69), which may be favorably affected by ACE inhibitors. ACE inhibitors also lower aldosterone secretion and renal potassium wasting, which could preserve β-cell responsiveness. ACE inhibitors may increase islet blood flow and pancreatic β-cell perfusion by reducing angiotensin-2 mediated vasoconstriction in the pancreas (Diabetologica. 1998; 41127-133). These effects may potentially slow or reverse the decline in β-cell function.

ACE inhibitors may reduce insulin resistance in skeletal muscles (, increase insulin-mediated glucose disposal thereby decreasing the need for pancreatic insulin secretion. The increased insulin mediated glucose uptake by skeletal muscle in response to an ACE inhibitor is due to increased bradykinin-mediated nitric oxide production and not to reductions in angiotensin 2 production or action (Am J. Physiol. 1999; 277:R332-R336; Br J Clin Pharmacol 1998; 46: 467-471). Several observations suggest that agents that increase nitric oxide (such as ACE-inhibitors) may also increase insulin-mediated glucose uptake, which include that (1) both insulin-mediated vasodilation and skeletal muscle glucose metabolism are reduced in obese persons who do not have diabetes (i.e., individuals at risk for diabetes) and in individuals with type 2 diabetes, (2) inhibition of nitric oxide production reproduces this effect in lean individuals, and (3) the effect on insulin sensitivity is greater than can be accounted for by just increased skeletal muscle blood flow (Am J Cardiol. 1999, 84: 25J-27J). ACE inhibitors may also reduce insulin resistance at the liver and fat cell, which would reduced hepatic glucose production and lower free fatty acid levels (Diabetolologia, 1991; 34: 119-125).

Implications

The data suggesting that ramipril, an ACE inhibitor, reduces the risk of developing diabetes mellitus require confirmation because of the enormous clinical and public health potential of these findings. We are therefore embarking on a large prospective trial (Diabetes Reduction Assessment Ramipril and rosiglitizone Medication [DREAM] among individuals with impaired glucose tolerance to evaluate prospectively whether ramipril prevents diabetes.

### SUMMARY

Context: Type 2 diabetes is a growing clinical and public health problem. Preventive efforts related to lifestyle modification are not always successful; therefore, alternative prevention strategies need to be studied.

Objective: To investigate the effectiveness of ramipril, an angiotensin-converting enzyme inhibitor, in preventing diabetes among high-risk persons.

Design, Setting and Participants: The randomized, controlled Heart Outcomes Prevention Evaluation trial of 5720 patients older than 55 years without known diabetes but with vascular disease who were followed up for a mean of 4.5 years. The study included 267 hospitals in 19 countries and was conducted between 1994 and 1999.

Intervention: Patients were randomly assigned to receive ramipril, up to 10 mg/d (n=2837), or placebo (n=2883).

Main Outcome Measure: Diagnosis of diabetes determined from self-report at follow-up visits every 6 months, compared between the 2 groups.

Results: One hundred and two individuals (3.6%) in the ramipril group developed diabetes compared with 155 (5.4%) in the placebo group (relative risk [RR], 0.66-95% confidence interval [Cl], 0.51-0.85, P<0.001). Similar results were noted when different diagnostic criteria were used; in the ramipril group, the RR for diagnosis of diabetes and hemoglobin A_{1c} greater than 110% was 0.60 (95% Cl, 0.43-085), for initiation of glucose-lowering therapy, 0.56 (95% Cl, 0.41-0.77), and for both, 0.51 (95% Cl, 0.34-0.76). These effects were also consistently seen in several subgroups examined.

### CONCLUSIONS

Ramipril is associated with lower rates of new diagnosis of diabetes in high-risk individuals. Because these results have important clinical and public health implications, this hypothesis requires prospective confirmation.

The contents of all patents, patent applications, published articles, books, reference manuals and abstracts cited herein are hereby incorporated by reference in their entirety to more fully describe the state of the art to which the invention pertains.

1. A method of preventing the development of diabetes in a patient who is at risk for developing diabetes, said method comprising administering to a patient, who is at risk for developing diabetes, an effective amount of an angiotensin converting enzyme inhibitor for a sufficient period of time to prevent the development of diabetes in such patient.

2. A method according to claim 1, wherein the diabetes is Type 2 Diabetes.

3. A method according to claim 1, wherein the angiotensin converting enzyme inhibitor is ramipril.

4. A method according to claim 2, wherein the angiotensin converting enzyme inhibitor is ramipril.

5. A method of preventing, slowing or reversing the decline of β-cell function in an individual comprising administering to an individual an effective amount of an angiotensin converting enzyme inhibitor for a sufficient period of time to prevent the decline of β-cell function in such individual.

6. A method according to claim 5, wherein the angiotensin converting enzyme inhibitor is ramipril.

7. A method of increasing islet blood flow in an individual comprising:
administering to an individual an effective amount of an angiotensin converting enzyme inhibitor for a sufficient period of time to increase islet blood flow in such individual.

8. A method according to claim 7, wherein the angiotensin converting enzyme inhibitor is ramipril.

9. A method of increasing pancreatic β-cell perfusion in an individual comprising:
administering to an individual an effective amount of an angiotensin converting enzyme inhibitor for a sufficient period of time to increase pancreatic β-cell perfusion in such individual.

10. A method according to claim 9, wherein the angiotensin converting enzyme inhibitor is ramipril.

11. A method of lowering aldosterone secretion and renal potassium wasting in an individual by comprising administering to an individual an effective amount of an angiotensin converting enzyme inhibitor for a sufficient period of time to lower aldosterone secretion and renal potassium wasting in such individual.

12-23. (Canceled)

24. A method according to claim 11, wherein the angiotensin converting enzyme inhibitor is ramipril.

25. A method of reducing diabetes in a patient, said method comprising administering to a patient an effective amount of an angiotensin converting enzyme inhibitor for a sufficient period of time to reduce diabetes in such patient.

26. A method according to claim 25, wherein the diabetes is Type 2 Diabetes.

27. A method according to claim 25, wherein the angiotensin converting enzyme inhibitor is ramipril.

28. A method according to claim 26, wherein the angiotensin converting enzyme inhibitor is ramipril.
